(19) United States
(12) Patent Application Publication
(10) Pub. No.: US 2026/0127797 A1
(43) Pub. Date: May 7, 2026

(54) FACIAL IMAGE DE-IDENTIFICATION METHOD AND SYSTEM

(71) Applicant: KNU-INDUSTRY COOPERATION FOUNDATION, Chuncheon-si (KR)

(72) Inventors: Woo Jin KIM, Chuncheon-si (KR); Kun Yong SUNG, Seoul (KR); Sang Won PARK, Chuncheon-si (KR); Seong UK KANG, Chuncheon-si (KR)

(73) Assignee: KNU-INDUSTRY COOPERATION FOUNDATION, Chuncheon-si (KR)

(21) Appl. No.: 19/194,239

(22) Filed: Apr. 30, 2025

(30) Foreign Application Priority Data
Nov. 5, 2024 (KR) 10-2024-0155485

(51) Int. Cl. G06T 11/00; G06T 15/08; G06V 10/74; G06V 10/774; G06V 40/16

(52) U.S. Cl. CPC G06T 12/30; G06T 15/08; G06V 10/761; G06V 10/7753; G06V 40/171; G06T 2210/41

(57) ABSTRACT

A facial image de-identification method and system are provided. A facial image de-identification method according to some embodiments may include acquiring a facial image, detecting one or more facial feature from the facial image, determining at least some of the detected facial features as a de-identification region of the facial image, and applying an image transformation technique to the determined de-identification region to generate a de-identification image. According to the method, a de-identification image can be created that preserves anatomical structure information such as a facial skeleton as it is while reducing the possibility of individual identification (that is, risk of re-identification).

### CROSS-REFERENCE TO RELATED APPLICATIONS

This application claims the priority of Korean Patent Application No. 10-2024-0155485 filed on Nov. 5, 2024, in the Korean Intellectual Property Office, the disclosure of which is incorporated herein by reference.

### BACKGROUND

### Field

The present disclosure relates to a technology for de-identifying a facial image.

### Description of the Related Art

Generally, when utilizing medical data within a hospital, a separate de-identification process is not required, but when exporting medical data to the outside for research collaboration with external institutions or multi-institutional joint research, the de-identification process is absolutely required to protect personal information of a patient.

Among various medical data, a computed tomography (CT) image is data that requires significant de-identification. This is because facial features such as the eyes, nose, mouth, and ears of the patient can be clearly restored from CT images, and these facial features can also be used to identify the patient.

Meanwhile, most existing facial image de-identification methods automatically remove a facial region from the facial image to reduce the possibility of individual identification. However, since the method also removes important structures such as the facial skeleton and teeth, the method has an obvious limitation in that the method cannot be utilized in fields (for example, plastic surgery research, or the like) that require anatomical structure information.

### PRIOR ART LITERATURE

### Patent Literature

(Patent Literature 1) Korean Patent Publication No. 10-2023-0080111 (Published on Jun. 7, 2023)

### SUMMARY

An object according to some embodiments of the present disclosure is to provide a de-identification method and system that may reduce the possibility of individual identification (that is, the risk of re-identification) while preserving overall structural information (for example, anatomical structure information such as facial skeleton, or the like) inherent in a facial image.

Objects of the present disclosure are not limited to the object mentioned above, and other objects not mentioned can be clearly understood by those skilled in the art in the technical field of the present disclosure from the description below.

In order to achieve the above-described object, according to some embodiments of the present disclosure, there is provided a facial image de-identification method performed by at least one processor, the method including: acquiring a facial image; detecting one or more facial features from the facial image; determining at least some of the detected facial features as a de-identification region of the facial image; and applying an image transformation technique to the determined de-identification region to generate a de-identification image.

In some embodiments, the image transformation technique may not be applied to a remaining region of the facial image except for the de-identification region.

In some embodiments, the one or more facial features may include at least one of an eye, a nose, a mouth, and an ear.

In some embodiments, the one or more facial features may include at least one of a scar and a birthmark.

In some embodiments, the facial image may be a tomographic image of a facial region.

In some embodiments, the detecting of the one or more facial features may include acquiring a deep learning model trained to detect the facial feature from an input image, and detecting the one or more facial features through the trained deep learning model.

In some embodiments, the training of the deep learning model may include acquiring a labeled image set and an unlabeled image set, the labeled image set being an image set to which a facial feature label is assigned, and the number of samples of the unlabeled image set being greater than that of the labeled image set, constructing an auxiliary deep learning model using the labeled image set, generating a training set by assigning the facial feature label to the unlabeled image set using the auxiliary deep learning model, and training the deep learning model using the training set.

In some embodiments, the method may further include: extracting a first feature embedding from the facial image through an image encoder; extracting a second feature embedding from the de-identification image through the image encoder; and calculating a re-identification risk score of the de-identification image based on similarity between the first feature embedding and the second feature embedding.

In some embodiments, the facial image may include slice images generated through tomography, and the de-identification image may include a plurality of de-identification slice images corresponding to the slice images, and the method may further include performing 3D volume rendering on the slice images to generate a first rendering image, performing the 3D volume rendering on the de-identification slice images to generate a second rendering image, extracting a first feature embedding from the first rendering image and a second feature embedding from the second rendering image through the image encoder, and calculating a re-identification risk score of the de-identification image based on similarity between the first feature embedding and the second feature embedding.

In some embodiments, wherein a plurality of facial features may be detected, and the determining of the detected facial feature as the de-identification region of the facial image may include generating a plurality of de-identification candidate combinations from the plurality of facial features, generating temporary de-identification images by applying the image transformation technique to each of the de-identification candidate combinations, calculating a re-identification risk score of each of the temporary de-identification images, selecting, among the de-identification candidate combinations, a de-identification candidate combination whose re-identification risk score is less than a reference value and satisfies preset conditions as a de-identification target combination, and determining the de-identification region based on the de-identification target combination. In this case, the preset condition may be defined based on at least one of the number of facial features and a region size belonging to a de-identification candidate combination.

In some embodiments, the detected facial feature may include a first facial feature and a second facial feature, the de-identification region may include the first facial feature, and the generating of the de-identification image may include generating an intermediate de-identification image by applying a first image transformation technique to the first facial feature, calculating a re-identification risk score of the intermediate de-identification image, adding the second facial feature to the de-identification region in response to a determination that the re-identification risk score is equal to or more than a reference value, and generating the de-identification image by applying a second image transformation technique to the second facial feature.

In order to achieve the above-described object, according to some embodiments of the present disclosure, there is provided a facial image de-identification system including: one or more processors; and a memory storing a computer program executed by the one or more processors, in which the computer program includes instructions for acquiring a facial image, detecting one or more facial features from the facial image, determining at least some of the detected facial features as a de-identification region of the facial image, and applying an image transformation technique to the determined de-identification region to generate a de-identification image.

In order to achieve the above-described object, according to some embodiments of the present disclosure, there is provided a computer program stored on a computer-readable recording medium, coupled with a processor of a computer, to execute: acquiring a facial image; detecting one or more facial features from the facial image; determining at least some of the detected facial features as a de-identification region of the facial image; and applying an image transformation technique to the determined de-identification region to generate a de-identification image.

According to some embodiments of the present disclosure, one or more facial features can be detected from the facial image, and at least some of the detected facial features can be determined as the de-identification region. Then, by applying the image transformation technique (that is, performing de-identification processing) to the de-identification region, the de-identification image corresponding to the facial image can be generated. That is, by performing local de-identification processing only on major facial features, the de-identification image can be generated. In this case, the de-identification image can be generated in which the possibility of individual identification (that is, the risk of re-identification) is low, while anatomical structure information such as facial skeleton is also preserved.

The effects according to the technical idea of the present disclosure are not limited to the effects mentioned above, and other effects not mentioned can be clearly understood by those skilled in the art from the description below.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is an exemplary diagram for explaining an operation of a de-identification system according to some embodiments of the present disclosure at a system level;

FIG. 2 is an exemplary diagram for further explaining the operation of the de-identification system according to some embodiments of the present disclosure;

FIG. 3 is an exemplary flowchart illustrating a facial image de-identification method according to some embodiments of the present disclosure;

FIG. 4A and FIG. 4B are exemplary diagrams for explaining a model construction method according to some embodiments of the present disclosure;

FIG. 5 illustrates a facial computer tomography (CT) image and a corresponding de-identification image that may be referenced in some embodiments of the present disclosure;

FIG. 6 illustrates a 3D volume rendering image that may be referenced in some embodiments of the present disclosure;

FIG. 7 is an exemplary diagram for explaining a re-identification risk score calculation method according to some embodiments of the present disclosure;

FIG. 8 is an exemplary diagram for explaining the results of a performance experiment conducted by the inventors of the present disclosure; and

FIG. 9 illustrates an exemplary computing device that may implement a de-identification system according to some embodiments of the present disclosure.

### DETAILED DESCRIPTION OF THE EMBODIMENT

Hereinafter, the exemplary embodiment of the present disclosure will be described with reference to the accompanying drawings and exemplary embodiments as follows. Scales of components illustrated in the accompanying drawings are different from the real scales for the purpose of description, so that the scales are not limited to those illustrated in the drawings.

Hereinafter, various embodiments of the present disclosure will be described in detail with reference to the attached drawings. The advantages and features of the present disclosure, and the methods for achieving them, will become clear with reference to the embodiments described in detail below together with the attached drawings. However, the technical idea of the present disclosure is not limited to the embodiments below, but can be implemented in various different forms, and the embodiments below are provided only to complete the technical idea of the present disclosure and to fully inform those with ordinary knowledge in the technical field to which the present disclosure belongs of the scope of the present disclosure, and the technical idea of the present disclosure is defined only by the scope of the claims.

In describing various embodiments of the present disclosure, when it is determined that a specific description of a related known configuration or function may obscure the gist of the present disclosure, the detailed description thereof will be omitted.

Unless otherwise defined, terms (including technical and scientific terms) used in the embodiments below may be used in a meaning that can be commonly understood by those with ordinary knowledge in the technical field to which the present disclosure belongs, but this may vary depending on the intention of a technician engaged in the relevant field, precedents, the emergence of new technologies, or the like. The terminology used in this disclosure is for the purpose of describing embodiments and is not intended to limit the scope of this disclosure.

In the following embodiments, the singular expression used includes the plural concept unless the context clearly specifies that it is singular. In addition, the plural expression includes the singular concept unless the context clearly specifies that it is plural.

In addition, the terms first, second, A, B, (a), (b), or the like used in the following embodiments are only used to distinguish one component from another, and the nature, order, or sequence of the corresponding component is not limited by the terms.

The components described with reference to the terms such as portion, unit, module, block, ˜or, ˜er, or the like used in the following embodiments and the functional blocks illustrated in the drawings may be implemented in the form of software, hardware, or a combination thereof. The software may be, for example, machine code, firmware, embedded code, and application software. In addition, the hardware may include, for example, electrical circuits, electronic circuits, processors, computers, integrated circuits, integrated circuit cores, passive components, or a combination thereof.

Hereinafter, various embodiments of the present disclosure will be described in detail with reference to the attached drawings.

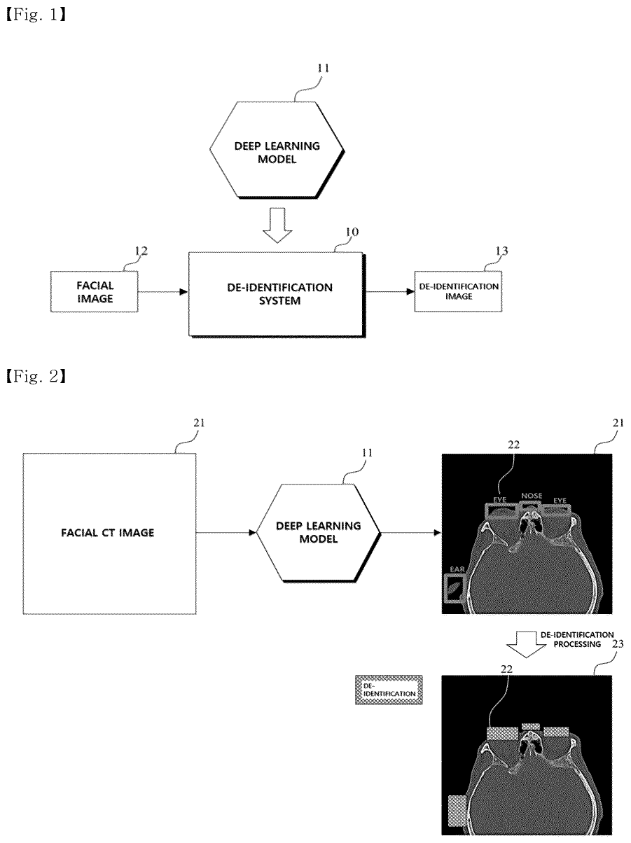

FIG. 1 is an exemplary diagram for explaining the operation of a de-identification system 10 according to some embodiments of the present disclosure at a system level.

As illustrated in FIG. 1, the de-identification system 10 is a computing device/system having a de-identification function for a facial image 12. For example, the de-identification system 10 may perform de-identification processing in a way that significantly reduces the possibility of individual identification (that is, the risk of re-identification) while preserving the overall structural information (for example, anatomical structure information such as a facial skeleton, or the like) inherent in the facial image 12, thereby generating a de-identification image 13 corresponding to the facial image 12. This de-identification system 10 may be named as a “facial image de-identification system” in some cases.

The facial image 12 is an original image that is a target of de-identification and may include various images regarding the facial area without limitation. In addition, when at least a part of the facial area is included, an image of the head area (that is, a head image) may also be included in the scope of the facial image 12. Examples of the facial image 12 may include a general image of the facial area and a tomographic image that contains anatomical structure information such as a facial (or head) skeleton, but the scope of the present disclosure is not limited thereto. In addition, examples of the tomographic image may include a computed tomography (CT) image, a magnetic resonance imaging (MRI) image, and a positron emission tomography (PET) image, but the scope of the present disclosure is not limited thereto. When the facial image 12 is a tomographic image, the facial image 12 may refer to a single slice (cross-section) image or may refer to a plurality of slice images.

Specifically, the de-identification system 10 may detect one or more facial features from the facial image 12 through a deep learning model 11 and perform the de-identification processing on at least some of the detected facial features, thereby generating a de-identification image 13 corresponding to the facial image 12. That is, the de-identification system 10 may de-identify all of the detected facial features or selectively de-identify some of the detected facial features.

In some cases, the de-identification system 10 may detect the facial features in the facial image 12 using a computer vision algorithm unrelated to the deep learning model 11.

The facial features may include, for example, eyes, nose, mouth, ears, scars, birthmarks, and other skin defects (for example, wrinkles, acne, pigmentation, skin lesions such as rashes, or the like). However, the scope of the present disclosure is not limited thereto.

In addition, the de-identification processing means processing an original image in a way that reduces the possibility of individual identification (that is, the risk of re-identification) by applying any image transformation technique.

Examples of such image transformation techniques may include mosaic processing, masking, blurring, adding noise, degradation (for example, down sampling), color change, shape deformation, and application of a substitute image, but the scope of the present disclosure is not limited thereto.

The deep learning model 11 refers to a model trained to detect the facial features from the input image, and may be named as a “facial feature detection (extraction) model” in some cases. This deep learning model 11 may be constructed using an image set to which facial feature labels (for example, labels including class and bounding box information, or labels including pixel-level class information) are assigned. The deep learning model 11 may be a model that detects facial features (that is, objects) based on bounding boxes, for example, but the scope of the present disclosure is not limited thereto. For example, the deep learning model 11 may detect the facial features through a semantic segmentation task.

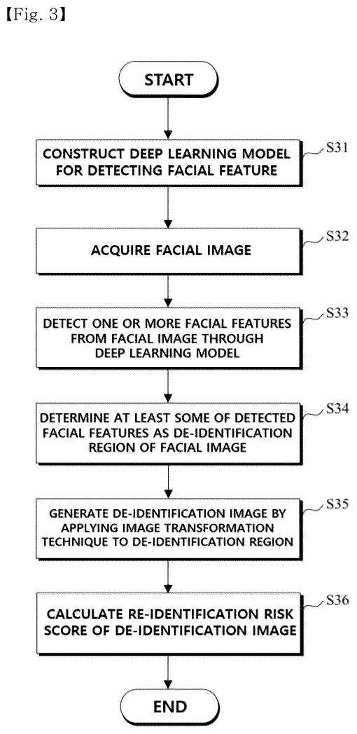

For better understanding, the operation of the de-identification system 10 will be described in more detail with reference to FIG. 2. FIG. 2 illustrates an example in which the de-identification target image is a CT image 21 of the facial area (more precisely, the head).

As illustrated in FIG. 2, the de-identification system 10 may detect facial features (for example, 22) such as eyes, nose, mouth, and ears from the facial CT image 21 through the deep learning model 11 (refer to “bounding box”), and apply an image transformation technique such as masking to the detected facial features (for example, 22). By doing so, a de-identification image 23 in which the overall anatomical structure information such as the facial skeleton is well preserved can be generated. In other words, by performing local de-identification processing on the facial features (for example, 22), the overall anatomical structure information inherent in the facial CT image 21 may be well preserved, and the risk of re-identification may be effectively reduced.

In some cases, the de-identification system 10 may selectively apply the image transformation technique to only some of the detected facial features (for example, 22).

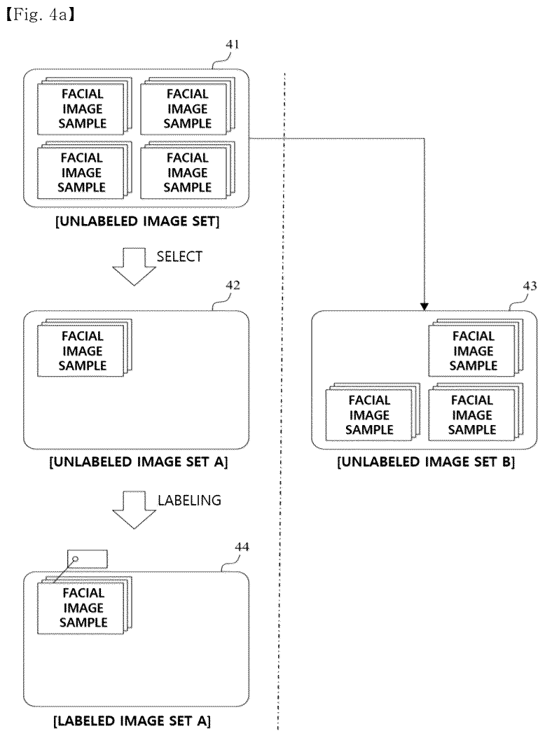

More specific details on the operation of the de-identification system 10 will be described in detail later with reference to drawings such as FIG. 3 and below.

The above-described de-identification system 10 may be implemented in at least one computing device. For example, all functions of the de-identification system 10 may be implemented in one computing device, or a first function of the de-identification system 10 may be implemented in a first computing device and a second function may be implemented in a second computing device. Alternatively, a specific function of the de-identification system 10 may be implemented in a plurality of computing devices.

The computing device may include any device having computing capabilities, and for an example of such a device, see FIG. 9. Since the computing device is a collection of interacting components (for example, memory, processor, or the like), the computing device may sometimes be called a “computing system”. Of course, the term such as the computing system may also encompass the concept of a collection of interacting computing devices.

So far, the operation of the de-identification system 10 according to some embodiments of the present disclosure has been briefly described with reference to FIGS. 1 and 2. Hereinafter, various methods that may be performed in the above-described de-identification system 10 will be described with reference to drawings including FIG. 3 and below.

Hereinafter, for the convenience of understanding, the explanation will be continued assuming that all steps/operations of the methods to be described later are performed in the de-identification system (10, for example, at least one processor) described above. Accordingly, when the subject of a specific step/operation is omitted, the step/operation can be understood as being performed by the de-identification system 10. However, in an actual environment, some steps/operations of the methods to be described later may be performed in other computing devices. For example, the construction (training) of the deep learning model 11, or the like may be performed in other computing devices/systems.

Hereinafter, for convenience of explanation, the de-identification system 10 will be abbreviated as a “system”.

FIG. 3 is an exemplary flowchart illustrating a facial image de-identification method according to some embodiments of the present disclosure. However, this is only an exemplary embodiment for achieving the purpose of the present disclosure, and it is to be understood that some steps may be added or deleted as needed.

As illustrated in FIG. 3, the present embodiments may start at Step S31 of constructing the deep learning model 11 for detecting facial features. For example, the system 10 may construct (train) a deep learning model 11 through supervised learning using an image set (that is, a labeled image set) to which a facial feature label is assigned. However, the specific construction method may vary depending on the embodiment.

In some embodiments, the system 10 may construct the deep learning model 11 through a bounding box-based object detection task. For example, the system 10 may construct the deep learning model 11 by fine-tuning a pre-trained object detection model (for example, YOLO, or the like) using the image set to which the facial feature label (that is, a label including class and bounding box information) is assigned.

In some other embodiments, the system 10 may construct the deep learning model 11 through a semantic segmentation task. For example, the system 10 may construct the deep learning model 11 by performing training using the image set to which the facial feature label (that is, label including pixel-level class information) is assigned or by fine-tuning a pre-trained semantic segmentation model.

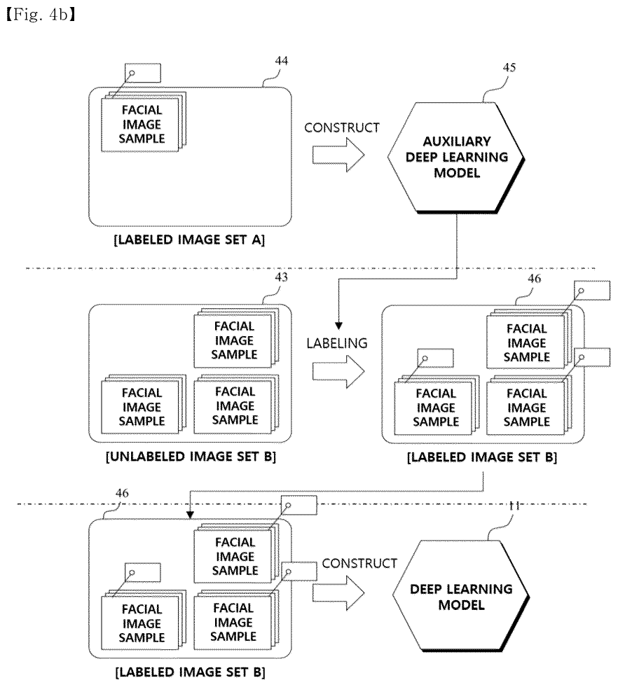

In some other embodiments, the system 10 may first construct an auxiliary deep learning model (hereinafter, abbreviated as “auxiliary model”) for a labeling task, and may generate a training set (that is, a labeled image set) by assigning the label to an unlabeled image set using the auxiliary model. Then, the system 10 may construct (train) the deep learning model 11 using the training set. By doing so, the time cost and human cost required for the labeling (annotation) task may be significantly reduced. These embodiments will be described in more detail with reference to FIGS. 4A and 4B below.

FIGS. 4A and 4B are exemplary diagrams for explaining a model construction method according to some embodiments of the present disclosure. Hereinafter, when elements distinguished by capital letters A and B in the drawings are referred to separately, modifiers (delimiters) such as “first” and “second” are used (however, this description rule is applied only when reference numbers exist).

As illustrated in FIG. 4A, first, the system 10 may select some facial image samples from an unlabeled image set 41 to form a first unlabeled image set 42. In this case, the number of samples in the first unlabeled image set 42 may be less than that in the second unlabeled image set 43 formed from the remaining samples.

The specific method of selecting facial image samples may be designed in various ways.

For example, the system 10 may extract feature embedding from each facial image sample of the unlabeled image set 41 and cluster the feature embeddings to construct a plurality of clusters. Then, the system 10 may select facial image samples in a balanced manner (that is, at the same or similar ratio) for each cluster. In this case, the system 10 may select the facial image sample from each of the center and the periphery of the cluster. By doing so, the facial image samples that can effectively train the auxiliary model 45 may be selected.

As another example, the system 10 may select the facial image sample having an entropy value equal to or more than a reference value from the unlabeled image set 41.

As another example, the system 10 may perform super resolution (SR) on each facial image sample of the unlabeled image set 41 and select the facial image sample whose SR result quality is equal to or less than the reference value.

As another example, the system 10 may select the facial image sample based on various combinations of the examples described above.

Next, the system 10 may receive label information on the facial feature from a user and generate a first labeled image set 44 from the first unlabeled image set 42. That is, the first labeled image set 44 may be generated through the manual labeling task.

Next, as illustrated in FIG. 4B, the system 10 may construct the auxiliary model 45 using the first labeled image set (44, that is, training set). This auxiliary model 45 may be named as a “labeling (annotation) model” in some cases.

Next, the system 10 may automatically perform the labeling task for the second unlabeled image set 43 using the auxiliary model 45. That is, the system 10 may assign the facial feature label (for example, class and bounding box information) to each of the facial image samples of the second unlabeled image set 43 using the auxiliary model 45. As a result, the second labeled image set 46, which is a training set of the deep learning model 11, is generated, and the time cost and human cost required for the labeling task may be significantly reduced.

Next, the system 10 may construct the deep learning model 11 using the second labeled image set 46. That is, the system 10 may construct the deep learning model 11 equipped with facial feature detection capability by performing supervised learning (that is, training) using the second labeled image set 46.

This will be described again with reference to FIG. 3.

In Step S32, the facial image is acquired. Here, the facial image means an original image to be de-identified, and the original means a state before de-identification. The method of acquiring the facial image may be any method.

As described above, the facial image may be a tomographic image containing overall anatomical structure information such as a facial skeleton, but the scope of the present disclosure is not limited thereto.

In Step S33, one or more facial features are detected (extracted) from the facial image through the deep learning model 11. For example, the system 10 may input the facial image into the deep learning model 11 to detect the facial features such as eyes, nose, mouth, and ears.

In some cases, the system 10 may detect one or more facial features using a computer vision algorithm unrelated to the deep learning model 11.

In Step S34, at least some of the detected facial features are determined as a de-identification region of the facial image. For example, the system 10 may determine all of the detected facial features (that is, all of the facial feature regions) as the de-identification region, or may determine some of the detected facial features as the de-identification region.

In some embodiments, the system 10 may generate a plurality of de-identification candidate combinations (for example, eyes, eyes and ears, eyes and mouth and ears, or the like) based on facial features detected from a facial image. Then, the system 10 may apply (that is, perform de-identification processing) the image transformation technique to each of the de-identification candidate combinations to generate temporary de-identification images, and may calculate a re-identification risk score for each of the temporary de-identification images. For a specific method of calculating the re-identification risk score, refer to the description of Step S36. Then, the system 10 may select the de-identification candidate combination having the re-identification risk score less than the reference value and satisfying a preset condition as a de-identification target combination among the de-identification candidate combinations. Here, the preset condition may be defined based on at least one of the number of facial features and the region size, and may be defined as, for example, when the number of facial features belonging to the de-identification candidate combination is less than (or minimum) the reference value, and when the region size of the facial feature is less than (or minimum) the reference value. Next, the system 10 may determine the de-identification region of the facial image based on the selected de-identification target combination (for example, the de-identification target combination having a sufficiently low re-identification risk score and the smallest number of facial features is determined as the de-identification region). In this case, the de-identification region may be determined in a way that the overall structural information of the facial image is preserved to the greatest extent while the re-identification risk is sufficiently low.

In Step S35, the de-identification image corresponding to the facial image is generated by applying the image transformation technique to the de-identification region (that is, performing de-identification processing). For example, the system 10 may generate the de-identification image by applying the image transformation technique only to the de-identification region (that is, performing local de-identification processing) without applying the image transformation technique to the remaining region except for the de-identification region. As described above, the image transformation technique may be, for example, mosaic processing, masking, or the like.

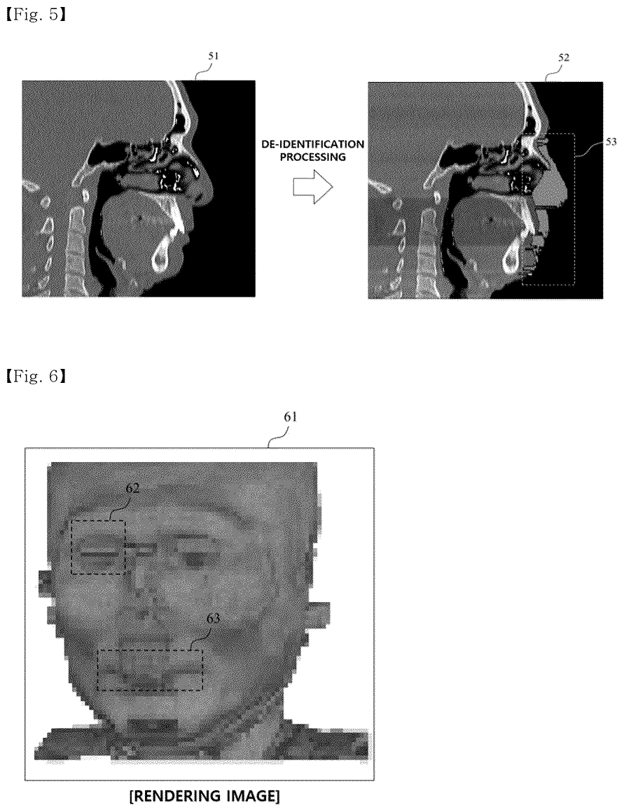

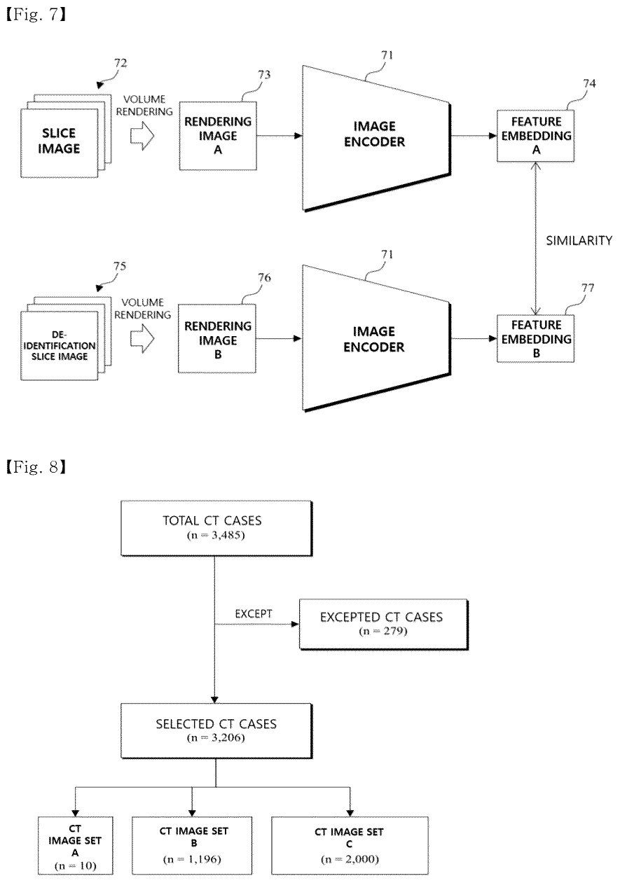

FIG. 5 illustrates an example of a result 52 of de-identification processing performed on a facial CT image 51 (more precisely, a slice image).

As illustrated in FIG. 5, when a local region 53 occupied by the facial features detected in the facial CT image 51 is determined as a de-identification region 53, the overall structural information such as the facial skeleton may be preserved as it is in the de-identification image 52.

FIG. 6 illustrates a result 61 of performing 3-dimensional volume rendering on the plurality of de-identification slice images. That is, FIG. 6 illustrates the result 61 of performing the 3D volume rendering after performing de-identification processing on each of the plurality of slice images constituting the facial CT image.

Referring to FIG. 6, when the de-identification processing is performed on the facial features such as eyes, nose, and mouth, information on major facial areas (see 62, 63) with strong identification power is removed, and thus, it can be confirmed that individual identification becomes significantly difficult even through the 3D volume rendering. That is, it can be confirmed that the risk of re-identification is effectively reduced by de-identification processing only on the facial features.

Meanwhile, in some embodiments, the system 10 may gradually increase the number of facial features to be de-identified until the re-identification risk score becomes less than the reference value. By doing so, the de-identification image in which overall facial structure information is preserved as much as possible may be generated. Specifically, assume that facial features detected from the facial image include a first facial feature and a second facial feature. In this case, the system 10 may include the first facial feature in the de-identification region of the facial image, and apply an image transformation technique (for example, masking) to the first facial feature (that is, perform de-identification processing) to generate the intermediate de-identification image. Then, the system 10 may calculate a re-identification risk score of the intermediate de-identification image. When the re-identification risk score is equal to or more than the reference value, the system 10 may add the second facial feature to the de-identification region of the corresponding facial image (that is, the de-identification region may be gradually expanded) and apply the image transformation technique (for example, masking) to the second facial feature. The system 10 may repeat this process until the re-identification risk score becomes less than the reference value.

This is explained again with reference to FIG. 3.

In Step S36, the re-identification risk score of the de-identification image is measured. This Step S36 may be omitted in some cases.

The specific method for calculating the re-identification risk score may vary depending on the embodiment.

In some embodiments, the re-identification risk score of the de-identification image may be calculated based on the difference in entropy between the facial image (that is, the original image) and the de-identification image. For example, the re-identification risk score may be calculated as a value inversely proportional to the difference in entropy.

In some other embodiments, the re-identification risk score of the de-identification image may be calculated based on a feature-level similarity between the facial image and the de-identification image. Using the feature-level similarity, the re-identification risk score may be calculated more accurately than the image-level similarity. Specifically, the system 10 may extract the first feature embedding (for example, one or more feature embeddings) from the facial image through an image encoder (for example, a pre-trained image encoder such as VGG16, DeepFace, or the like), and extract a second feature embedding (for example, one or more feature embeddings) from the de-identification image. Then, the system 10 may calculate the re-identification risk score of the de-identification image based on the similarity (for example, cosine similarity, or the like) between the first feature embedding and the second feature embedding (for example, the re-identification risk score is calculated as a value proportional to the similarity). For example, the system 10 may extract a first feature embedding set including the plurality of feature embeddings from the facial image, and extract a second feature embedding set from the de-identification image. Then, the system 10 may calculate a re-identification risk score of the de-identification image based on the similarity between the first feature embedding set and the second feature embedding set. In this case, the re-identification risk score may be calculated more accurately by comparing the feature embeddings in various aspects.

For reference, the term such as the image encoder may sometimes be named as “visual encoder” or “feature extractor”, and the term such as the feature embedding may sometimes be named as “feature”, “feature representation”, “feature vector”, “embedding representation”, “embedding vector”, or “embedding”.

In some other embodiments, the re-identification risk score of the de-identification image may be calculated based on the entropy difference between rendering images generated through the 3D volume rendering. For example, assume that the facial image is a tomographic image including the plurality of slice images. In this case, the system 10 may perform the 3D volume rendering on the plurality of slice images to generate the first rendering image, and also perform 3D volume rendering on a plurality of de-identification slice (that is, images obtained by images performing de-identification processing on each of the plurality of slice images) to generate a second rendering image. Then, the system 10 may calculate the re-identification risk score of the de-identification image (for example, at least one image among the plurality of de-identification slice images) based on the entropy difference between the first rendering image and the second rendering image.

In some other embodiments, the re-identification risk score of the de-identification image may be calculated based on the feature-level similarity between rendering images generated through the 3D volume rendering. For example, as illustrated in FIG. 7, assume that a facial image 72 is a tomographic image including the plurality of slice images. In this case, the system 10 may perform the 3D volume rendering on the plurality of slice images 72 to generate a first rendering image 73. Then, the system 10 may extract a first feature embedding 74 from the first rendering image 73 through an image encoder 71. Then, the system 10 may perform the 3D volume rendering on a plurality of de-identification slice images 75 (that is, images obtained by performing de-identification processing on each of the plurality of slice images 72) to generate a second rendering image 76. Next, the system 10 may extract a second feature embedding 77 from the second rendering image 76 through the image encoder 71. Next, the system 10 may calculate the re-identification risk score of the de-identification image (for example, at least one image among the de-identification slice images 75) based on the similarity between the first feature embedding 74 and the second feature embedding 77.

In some other embodiments, the re-identification risk score of the de-identification image may be calculated based on various combinations of the above-described embodiments. For example, the system 10 may calculate a first re-identification risk score based on the entropy difference between the facial image and the de-identification image, calculate a second re-identification risk score based on the similarity between the feature embeddings, and calculate a final re-identification risk score through a weighted sum between the first re-identification risk score and the second re-identification risk score.

Meanwhile, when the calculated re-identification risk score is equal to or more than the reference value (that is, when the re-identification risk is evaluated to be high), the system 10 may perform additional de-identification processing. For example, when there is a facial feature that is not included in the de-identification region, the system 10 may add the facial feature to the de-identification region and apply the image transformation technique (that is, perform de-identification processing). As another example, the system 10 may apply a stronger image transformation technique than before to a specific facial feature. As another example, the system 10 may designate one or more regions in the de-identification image (for example, randomly designate, designate a region close to a facial feature, or the like) and add the designated region to the de-identification region (that is, de-identification processing is also performed on the designated region). The illustrated operations may be repeatedly performed until the re-identification risk score becomes lower than the reference value.

Hereinafter, the facial image de-identification method according to some embodiments of the present disclosure will be described with reference to FIGS. 3 to 7. As described above, one or more facial features may be detected from a facial image, and at least some of the detected facial features may be determined as the de-identification region. Then, the image transformation technique may be applied to the de-identification region to generate the de-identification image corresponding to the facial image. That is, the de-identification image may be generated by performing local de-identification processing only on major facial features. In this case, the de-identification image may be generated in which the possibility of individual identification (that is, risk of re-identification) is low, while anatomical structure information such as facial skeleton is preserved as it is.

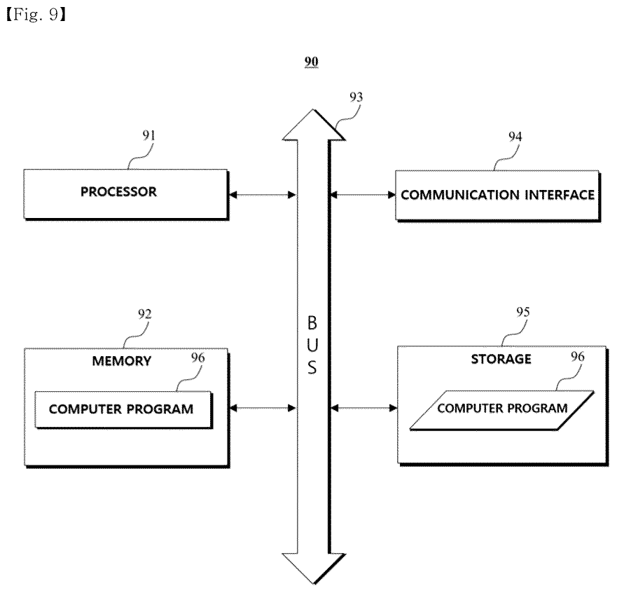

Below, the results of a performance experiment conducted by the inventors of the present disclosure are briefly introduced with reference to FIG. 8.

The present inventors conducted an experiment to evaluate (verify) the performance of the above-described facial image de-identification method (hereinafter, referred to as a “proposed method”).

Specifically, as illustrated in FIG. 8, the inventors collected a total of 3,485 CT cases regarding patients with facial fractures treated at a plastic surgery department (here, each CT case includes multiple slice images), and finally selected 3,206 CT cases by excluding some CT cases that did not meet the criteria. From the collected CT cases, the inventors excluded CT cases of patients under 18 years of age with immature facial skeletons and CT cases taken with other equipment.

Next, the inventors constructed three CT image sets A to C using the selected CT cases. The CT image set A was constructed in a smaller size than the CT image set B.

Next, the inventors manually assigned labels for eyes, nose, mouth, and ears to the CT image set A and used the labels to construct (train) a “deep learning model M1” corresponding to the auxiliary model. In addition, the inventors automatically assigned labels to the CT image set B using the deep learning model M1 and used the labels to construct (train) a deep learning model M2. For this, refer to the description of FIGS. 4A and 4B. “YOLOv8”, which was an object detection model based on bounding boxes, was used as the deep learning models M1 and M2.

Next, the inventors evaluated the performance of the proposed method using the CT image set C as a test set.

First, the inventors conducted an experiment to evaluate the performance (that is, facial feature detection performance) of the deep learning models M1 and M2 using the CT image set C. The performance metrics used were mean average precision (mAP), precision, recall, and F1-score, and the evaluation results are illustrated in Table 1 below.

TABLE 1

| Model | mAP_0.5 | mAP_0.5:0.95 | Precision | Recall | F1-score |
|---|---|---|---|---|---|
| M1 | 0.892 | 0.414 | 0.916 | 0.866 | 0.891 |
| M2 | 0.902 | 0.450 | 0.880 | 0.864 | 0.872 |

Referring to Table 1, the performance of the deep learning model M2 was generally better than that of the deep learning model M1. This confirms that the model construction method described in FIGS. 4A and 4B may effectively reduce labeling costs while constructing a deep learning model with excellent performance.

In addition, the inventors conducted an experiment to evaluate the re-identification success rate using the CT image set C and the deep learning model M2. Specifically, the inventors selected a specific CT facial image (for example, slice image, hereinafter referred to as “original image”) from the CT image set C and generated the de-identification image corresponding to the original image through the proposed method. Then, the inventors selected the top five ranks of CT facial images with high feature-level similarity to the de-identification image from the CT image set C and confirmed whether the CT facial image of the specific rank corresponds to the original image. The inventors repeated this process several times for each rank. The feature-level similarity (however, cosine similarity was used) was calculated in the manner illustrated in FIG. 8, and the evaluation results are described in Table 2 below.

TABLE 2

| Similarity top rank | 5 | 4 | 3 | 2 | 1 |
|---|---|---|---|---|---|
| Re-identification success | 29.18 | 26.91 | 24.44 | 20.88 | 15.83 |
| rate (%) | | | | | |

As illustrated in Table 2, the possibility of re-identifying the original image from the de-identification image generated by the proposed method was significantly lower. This confirms that the proposed method effectively reduces the risk of re-identification while preserving the overall structural information inherent in the facial image.

In addition, the inventors conducted a blind test targeting the general public and plastic surgeons to determine the original image corresponding (matching) to the de-identification image. Specifically, the inventors selected a specific CT facial image (for example, slice image, hereinafter referred to as “original image”) from the CT image set C and generated the de-identification image corresponding to the original image through the proposed method. Then, the inventors configured the de-identification image, the original image, and the top four rank CT facial images with high feature-level similarity with the de-identification image as pairs, and prepared the de-identification image set by generating multiple such pairs. Then, the inventors conducted a blind test targeting the general public and plastic surgeons to determine the original image from each pair of the de-identification image set. The results are illustrated in Table 3 below.

TABLE 3

| | De-identification image set |
|---|---|
| | (accuracy (%) ± standard |
| Group | deviation) |
| General public (10 people) | 55 ± 13 |
| Plastic surgeons (5 people) | 54 ± 13 |

Referring to Table 3, it was illustrated that the possibility of re-identifying the original image from the de-identification image generated by the proposed method is quite low. In particular, it was illustrated that even plastic surgeons had difficulty in distinguishing the original image corresponding to the de-identification image. This confirms once again that the proposed method effectively reduces the risk of re-identification while preserving the overall structural information inherent in the facial image.

So far, the results of performance experiments conducted by the inventors of the present disclosure have been briefly introduced with reference to FIG. 8. Hereinafter, an exemplary computing device 90 capable of implementing the above-described system 10 will be described with reference to FIG. 9.

FIG. 9 is an exemplary hardware configuration diagram illustrating the computing device 90.

As illustrated in FIG. 9, the computing device 90 may include one or more processors 91, a bus 93, a communication interface 94, a memory 92 for loading a computer program 96 executed by the processor 91, and a storage 95 for storing the computer program 96. However, only components related to the embodiment of the present disclosure are illustrated in FIG. 9. Therefore, a person skilled in the art to which the present disclosure pertains may understand that other general components may be further included in addition to the components 91 to 96 illustrated in FIG. 9. That is, the computing device 90 may further include various components in addition to the components 91 to 96 illustrated in FIG. 9. In addition, in some cases, the computing device 90 may be configured in a form in which some of the components 91 to 96 illustrated in FIG. 9 are omitted. Hereinafter, each component of the computing device 90 will be described.

The processor 91 may control the overall operation of each component of the computing device 90. The processor 91 may be configured to include at least one of a central processing unit (CPU), a micro processor unit (MCU), a micro controller unit (MCU), a graphic processing unit (GPU), a tensor processing unit (TPU), a neural processing unit (NPU), or any other type of processor well known in the art of the present disclosure. In addition, the processor 91 may perform operations for at least one application or program to execute specific steps/operations/methods. The computing device 90 may have one or more processors.

Next, the memory 92 may store various data, commands and/or information. The memory 92 may load a computer program 96 from the storage 95 to execute specific steps/operations/methods. The memory 92 may be implemented as a volatile memory such as RAM, but the technical scope of the present disclosure is not limited thereto.

Next, the bus 93 can provide a communication function between components of the computing device 90. The bus 93 may be implemented as various types of buses such as an address bus, a data bus, and a control bus.

Next, the communication interface 94 may support wired and wireless Internet communication of the computing device 90. In addition, the communication interface 94 may support various communication methods other than Internet communication. To this end, the communication interface 94 may be configured to include a communication module well known in the technical field of the present disclosure.

Next, the storage 95 can non-temporarily store one or more computer programs 96. The storage 95 may be configured to include a non-volatile memory such as a read only memory (ROM), an erasable programmable ROM (EPROM), an electrically erasable programmable ROM (EEPROM), and a flash memory, a hard disk, a removable disk, or any form of computer-readable recording medium well known in the art to which the present disclosure pertains.

Next, the computer program 96 may include instructions to cause the processor 91 to perform specific steps/operations/methods when loaded into the memory 92. That is, the processor 91 may perform specific steps/operations/methods by executing the instructions loaded into the memory 92.

For example, the computer program 96 may include instructions for acquiring the facial image, detecting one or more facial features from the facial image, determining at least some of the detected facial features as the de-identification region of the facial image, and applying the image transformation technique to the determined de-identification region to generate the de-identification image.

As another example, the computer program 96 may include instructions to perform at least some of the steps/operations/methods described with reference to FIGS. 1 to 8.

In the case illustrated, the system 10 according to some embodiments of the present disclosure may be implemented via the computing device 90.

Meanwhile, in some embodiments, the computing device 90 illustrated in FIG. 9 may mean a virtual machine implemented based on cloud technology. For example, the computing device 90 may be a virtual machine operating on one or more physical servers included in a server farm. In this case, at least some of the processor 91, memory 92, and storage 95 illustrated in FIG. 9 may be virtual hardware, and the communication interface 94 may also be implemented as a virtualized networking element such as a virtual switch.

Hereinafter, the exemplary computing device 90 capable of implementing a system 10 according to some embodiments of the present disclosure has been described with reference to FIG. 9.

Meanwhile, in order to help understanding of the present disclosure, additional explanations are as follows.

The de-identification processing system is designed to individually de-identify the facial features such as eyes, nose, mouth, and ears according to the necessity of the study. This system provides flexibility to harmoniously maintain the usability of data and privacy protection according to the purpose of the study.

The criteria for selecting facial features vary depending on the necessity of maintaining the usability of data.

For example, when the structure of the nose is important in a specific clinical study or analysis, a part of the nose is selectively de-identified so as not to damage the original purpose of the data.

Similarly, specific parts of the mouth or eyes may also be de-identified according to the purpose of the study.

In this way, by subdividing the de-identification processing and de-identifying the necessary parts, the value of the data may be preserved to the greatest extent possible. In addition, legal and regulatory requirements should be considered, and the level of the de-identification processing may be adjusted according to the privacy protection regulations of the country or institution where the research is being conducted.

Meanwhile, the decision on which facial features to de-identify in the facial de-identification process largely depends on the characteristics and purpose of the data required for the research.

When the research purpose is to analyze or preserve specific facial structures or anatomical information, the strategy of the de-identification processing should be carefully designed to ensure privacy protection while maintaining the usability of the data as much as possible.

For example, when the shape of the nose or the structure of the mouth plays an important role in clinical research or medical analysis, completely de-identifying these features may have a negative impact on the usability of the research data.

Therefore, in such studies, the entire structure of the nose or mouth may be preserved, and relatively less important parts such as the eyes or ears may be de-identified. This may reduce the possibility of individual identification while achieving the original research purpose of the data.

Meanwhile, there are several methods to choose from to measure the risk of re-identification of the de-identified CT images.

### 1. Face Embedding

Face Embedding is a method of converting facial images into high-dimensional vectors to quantify the facial features. These vectors contain unique biometric information about the face and are used to compare the similarity between different faces. Face recognition is performed by calculating the distance or similarity between the converted vectors.

For example, FaceNet, developed by Google, converts facial images into 128-dimensional vectors. It calculates the Euclidean distance between these vectors to determine whether the two faces are of the same person.

The shorter the distance, the higher the probability that the two faces are of the same person. ArcFace uses an angle-based loss function to further increase the accuracy of comparing facial vectors.

### 2. Siamese Network

Siamese Network is a method that uses two neural networks with the same structure for face recognition.

This network processes two facial 1 images to generate feature vectors for each, and then analyzes the differences between the vectors to determine whether the two faces are of the same person.

This method is very effective for verifying facial matching, and is especially used to increase the accuracy of face comparison.

Siamese Network is used to strengthen security authentication in facial recognition systems and provides high reliability in verifying whether a person's face is the same.

For example, Siamese Network may be applied to unlocking smartphones or security authentication in financial services.

### 3. Triplet Loss Function

The triplet loss function is a technique used to train the face embedding model.

In this method, three data of anchor, positive, and negative samples are used to train the difference between faces.

The goal is to minimize the distance between the anchor and positive samples and maximize the distance between the anchor and negative samples.

This process trains the model to distinguish subtle differences in facial features, thereby improving recognition accuracy.

In particular, the triplet loss function is used to more accurately determine whether it is the same person in facial recognition systems.

For example, the triplet loss function can be used to accurately identify faces in door control systems or to verify identities in important security systems.

Meanwhile, the system does not have a function to determine the level of de-identification by automatically determining the risk of re-identification, but instead, the system is designed so that the user can individually select the eyes, nose, mouth, and ears and perform de-identification according to the user's needs.

This provides the user with flexibility to adjust the level of de-identification.

For example, when the structure of the nose is important for the research purpose, the user may keep the nose without de-identification.

Meanwhile, when the risk of re-identification is a concern, the user may selectively de-identify the eyes or ears to increase the level of privacy protection.

This user-selection function supports researchers to maximize the usefulness of data while minimizing the possibility of individual identification when necessary.

This system does not use a fixed de-identification method, but provides a customized approach that allows the user to adjust the de-identification strategy according to the research purpose and situation.

This allows the user to maintain a balance between data usefulness and privacy protection, and to apply various de-identification scenarios according to the user's needs.

Meanwhile, this facial feature de-identification system targets 3D images from which faces may be restored during 3D reconstruction, and may be applied to medical images such as CT and MRI.

The 3D image referred to here is an image that is reconstructed into a three-dimensional structure by shooting individual slices and overlapping or connecting them.

When de-identifying medical images such as CT images and MRI images, the de-identification processing should be adjusted according to the unique characteristics and diagnostic purpose of each image.

Since both CT and MRI can shoot the human body as individual slices and then reconstruct the slices into 3D, the same de-identification process may be applied.

The purpose of de-identification in these 3D reconstructed medical images is to prevent the possibility of the face being identified again when facial features are individually removed from each slice and then combined into 3D.

Therefore, by applying the same de-identification process to both CT and MRI, and performing detailed adjustments according to the characteristics of each image, accurate de-identification and clinical usefulness of data may be secured at the same time.

CT images are a method of obtaining cross-sectional tomographic images by transmitting X-rays, and are mainly used to check the internal structure of bones or hard tissues.

For example, lung diseases such as lung cancer, inflammatory diseases of the lungs, and chronic bronchial diseases can be precisely diagnosed through CT examinations, and kidney and adrenal diseases, cancer, cancer, and liver stomach pancreatobiliary cancer can also be diagnosed through CT. CT equipment is distinguished by the number of channels such as “64, 128, and 256ch”, and the higher the number of channels, the more accurately and quickly a wide lesion can be examined.

Due to these characteristics, when de-identifying CT images, it is common to de-identify the main features of the face (eyes, nose, mouth, ears) excluding hard tissues such as bones.

However, when the bone structure is not important, additional facial deformation techniques can be applied.

MRI images use strong magnetic fields and high frequencies to capture detailed images of soft tissues, nerves, and muscles of the human body.

MRI provides more precise 3D images than CT, and can examine areas such as the brain, nerves, blood vessels, muscles, and ligaments in detail.

In particular, MRI can view both longitudinal and transverse sections, making it easy to interpret diseases from various angles.

MRI can better detect lesions in soft tissues that are difficult to find with CT, making it very effective in interpreting muscle ruptures, nerve damage, and disc problems.

However, when de-identifying MRI images, distinction between soft tissues and bones is more important than distinction between bones, so a technique for effectively de-identifying facial contours and soft tissue information is required.

Various embodiments of the present disclosure and effects according to the embodiments have been described with reference to FIGS. 1 to 9 so far. Effects according to the technical idea of the present disclosure are not limited to the effects mentioned above, and other effects not mentioned will be clearly understood by those skilled in the art from the description below.

In addition, even though the above embodiments have described multiple components as being combined or combined to operate as one, the technical idea of the present disclosure is not necessarily limited to these embodiments. That is, within the scope of the purpose of the technical idea of the present disclosure, all of the components may be selectively combined and operated as one or more.

The technical idea of the present disclosure described so far may be implemented as a computer-readable code on a computer-readable recording medium. A computer program stored in a computer-readable recording medium may be transmitted to another computing device through a network such as the Internet and installed in the computing device, thereby being used in the computing device.

Although the operations are illustrated in a specific order in the drawings, it should not be understood that the operations must be executed in the specific order illustrated or in a sequential order, or that all illustrated operations must be executed to obtain a desired result. In certain situations, multitasking and parallel processing may be advantageous. Although various embodiments of the present disclosure have been described with reference to the attached drawings, those skilled in the art to which the present disclosure pertains will understand that the technical ideas of the present disclosure can be implemented in other specific forms without changing the technical ideas or essential features thereof. Therefore, it should be understood that the embodiments described above are exemplary in all respects and not restrictive. The protection scope of the present disclosure should be interpreted by the following claims, and all technical ideas within a scope equivalent thereto should be interpreted as being included in the scope of rights of the technical ideas defined by the present disclosure.

What is claimed is:

1. A facial image de-identification method performed by at least one processor, the method comprising:
acquiring a facial image;
detecting one or more facial features from the facial image;
determining at least some of the detected facial features as a de-identification region of the facial image; and
applying an image transformation technique to the determined de-identification region to generate a de-identification image.

2. The method of claim 1, wherein the image transformation technique is not applied to a remaining region of the facial image except for the de-identification region.

3. The method of claim 1, wherein the one or more facial features include at least one of an eye, a nose, a mouth, and an ear.

4. The method of claim 1, wherein the one or more facial features include at least one of a scar and a birthmark.

5. The method of claim 1, wherein the facial image is a tomographic image of a facial region.

6. The method of claim 1, wherein the detecting of the one or more facial features includes
acquiring a deep learning model trained to detect the facial feature from an input image, and
detecting the one or more facial features through the trained deep learning model.

7. The method of claim 6, wherein the training of the deep learning model includes
acquiring a labeled image set and an unlabeled image set, the labeled image set being an image set to which a facial feature label is assigned, and the number of samples of the unlabeled image set being greater than that of the labeled image set,
constructing an auxiliary deep learning model using the labeled image set,
generating a training set by assigning the facial feature label to the unlabeled image set using the auxiliary deep learning model, and
training the deep learning model using the training set.

8. The method of claim 1, further comprising:
extracting a first feature embedding from the facial image through an image encoder;
extracting a second feature embedding from the de-identification image through the image encoder; and
calculating a re-identification risk score of the de-identification image based on similarity between the first feature embedding and the second feature embedding.

9. The method of claim 1, wherein the facial image includes a plurality of slice images generated through tomography, and
the de-identification image includes a plurality of de-identification slice images corresponding to the slice images, and
the method further includes:
performing 3D volume rendering on the slice images to generate a first rendering image;
performing the 3D volume rendering on the de-identification slice images to generate a second rendering image;
extracting a first feature embedding from the first rendering image and a second feature embedding from the second rendering image through the image encoder; and
calculating a re-identification risk score of the de-identification image based on similarity between the first feature embedding and the second feature embedding.

10. The method of claim 1, wherein the plurality of facial features is detected, and
the determining of the detected facial feature as the de-identification region of the facial image includes
generating a plurality of de-identification candidate combinations from the plurality of facial features,
generating temporary de-identification images by applying the image transformation technique to each of the de-identification candidate combinations,
calculating a re-identification risk score of each of the temporary de-identification images,
selecting, among the de-identification candidate combinations, a de-identification candidate combination whose re-identification risk score is less than a reference value and satisfies preset conditions as a de-identification target combination, and
determining the de-identification region based on the de-identification target combination, and
the preset condition is defined based on at least one of the number of facial features and a region size belonging to the de-identification candidate combination.

11. The method of claim 1, wherein the detected facial feature includes a first facial feature and a second facial feature,
the de-identification region includes the first facial feature, and
the generating of the de-identification image includes
generating an intermediate de-identification image by applying a first image transformation technique to the first facial feature,
calculating a re-identification risk score of the intermediate de-identification image,
adding the second facial feature to the de-identification region in response to a determination that the re-identification risk score is equal to or more than a reference value, and
generating the de-identification image by applying a second image transformation technique to the second facial feature.

12. A facial image de-identification system comprising:
one or more processors; and
a memory storing a computer program executed by the one or more processors,
wherein the computer program includes instructions for acquiring a facial image,
detecting one or more facial features from the facial image,
determining at least some of the detected facial features as a de-identification region of the facial image,
and
applying an image transformation technique to the determined de-identification region to generate a de-identification image.

13. The facial image de-identification system of claim 12, wherein the image transformation technique is not applied to a remaining region of the facial image except for the de-identification region.

14. The facial image de-identification system of claim 12, wherein the one or more facial features include at least one of an eye, a nose, a mouth, and an ear.

15. A computer program stored on a computer-readable recording medium, coupled with a processor of a computer, to execute:
acquiring a facial image;
detecting one or more facial features from the facial image;
determining at least some of the detected facial features as a de-identification region of the facial image;
and
applying an image transformation technique to the determined de-identification region to generate a de-identification image.
